(19) United States
(12) Patent Application Publication
(10) Pub. No.: US 2026/0182956 A1
(43) Pub. Date: Jul. 2, 2026

(54) METHODS AND SYSTEMS FOR ULTRASOUND IMAGE GENERATION

(71) Applicant: WUHAN UNITED IMAGING HEALTHCARE CO., LTD., Wuhan (CN)

(72) Inventor: Yunhao ZHU, Shanghai (CN)

(73) Assignee: WUHAN UNITED IMAGING HEALTHCARE CO., LTD., Wuhan (CN)

(21) Appl. No.: 19/437,220

(22) Filed: Dec. 30, 2025

(30) Foreign Application Priority Data
Dec. 30, 2024 (CN) 202411977957.1

(51) Int. Cl. A61B 8/14; A61B 8/00

(52) U.S. Cl. CPC A61B 8/14; A61B 8/483; A61B 8/543

(57) ABSTRACT

An ultrasound image generation method is provided. The method includes obtaining a plurality of ultrasonic echo signals of a subject. The plurality of ultrasonic echo signals are acquired based on receiving planes corresponding to different transmitting planes. The method includes obtaining a plurality of receiving plane estimations corresponding to the receiving planes and generating a plurality of ultrasonic echo signal estimations each of which corresponds to one of the plurality of receiving plane estimations based on the plurality of ultrasonic echo signals. The method includes generating a two-dimensional ultrasound image of each of the receiving plane estimations based on the plurality of ultrasonic echo signal estimations corresponding to the plurality of receiving plane estimations. The method also includes obtaining a three-dimensional ultrasound image of the subject based on two-dimensional ultrasound images of the plurality of receiving plane estimations.

### CROSS-REFERENCE TO RELATED APPLICATIONS

This application claims priority to the Chinese Patent Application No. 202411977957.1, filed on Dec. 30, 2024, the entire contents of which are hereby incorporated by reference.

### TECHNICAL FIELD

The present disclosure generally relates to a field of ultrasound detection, and in particular to a method and a system for an ultrasound image generation.

### BACKGROUND

Two important indicators of four-dimensional (4D) ultrasound imaging are imaging rate and imaging resolution. In related technologies, four-dimensional ultrasound imaging typically uses reconstruction based on 2D (two-dimensional) images or based on 3D (three-dimensional) images. Among them, the manner based on 2D reconstruction involves interpolating a plurality of 2D images into one 3D image. Since 2D image reconstruction is fast, it offers certain advantages in imaging rate. However, during the interpolation process of 2D images, the correlation between the various 2D planes cannot be taken into account, resulting in severe degradation and low image resolution of the reconstructed 3D image. The manner based on 3D reconstruction involves generating one 3D image by algorithmically processing all transmit-receive data. Since this algorithm fully considers the correlation of echo signals at various spatial positions, the imaging resolution is high. However, the computational load for 3D reconstruction is several times or even dozens of times greater than that for 2D reconstruction, resulting in a low imaging rate that cannot meet the requirements of four-dimensional ultrasound imaging.

Therefore, it is necessary to provide a method and system for ultrasound image generation to balance the imaging rate and imaging resolution of ultrasound imaging.

### SUMMARY

An aspect of the present disclosure provides an ultrasound image generation method. The method includes obtaining a plurality of ultrasonic echo signals of a subject. The plurality of ultrasonic echo signals are acquired based on one or more receiving planes corresponding to one or more transmitting planes. The method includes obtaining a plurality of receiving plane estimations corresponding to the one or more receiving planes and generating a plurality of ultrasonic echo signal estimations each of which corresponds to one of the plurality of receiving plane estimations based on the plurality of ultrasonic echo signals. The method includes generating a two-dimensional ultrasound image of each of plurality of receiving plane estimations based on the plurality of ultrasonic echo signal estimations corresponding to the plurality of receiving plane estimations. The method also includes obtaining a three-dimensional ultrasound image of the subject based on two-dimensional ultrasound images of the plurality of receiving plane estimations.

In some embodiments, the obtaining a plurality of receiving plane estimations corresponding to the one or more receiving planes includes determining the plurality of receiving plane estimations corresponding to each of the one or more receiving planes based on a configuration parameter of an ultrasound device.

In some embodiments, the determining the plurality of receiving plane estimations corresponding to each of the one or more receiving planes based on a configuration parameter of an ultrasound device includes determining, based on the configuration parameter, a second count of the plurality of receiving plane estimations corresponding to each of the one or more receiving planes and determining the plurality of receiving plane estimations corresponding to each of the one or more receiving planes based on the second count of the plurality of receiving plane estimations. The plurality of receiving plane estimations include a plurality of different virtual receiving planes sequentially arranged along two sides of a receiving plane corresponding to the each of the transmitting planes;

In some embodiments, the determining a second count of receiving plane estimations corresponding to each of the one or more receiving planes based on the configuration parameter includes determining an included angle between each of the plurality of different virtual receiving planes and the receiving plane based on an elevation angle spread of the receiving plane and the second count of the receiving plane estimations and determining the plurality of the virtual receiving planes arranged along the two sides of the receiving plane based on the included angle. The elevation angle spread represents an included angle between adjacent receiving planes.

In some embodiments, the determining a second count of the plurality of receiving plane estimations corresponding to each of the one or more receiving planes based on the configuration parameter includes in response to a volume of the subject being greater than a preset volume, increasing an initial second count of the plurality of receiving plane estimations by adjusting the configuration parameter to obtain the second count.

In some embodiments, the determining a second count of the plurality of receiving plane estimations corresponding to each of the one or more receiving planes based on the configuration parameter includes: in response to a heartbeat frequency of the subject being greater than a preset frequency, reducing an initial second count of the plurality of receiving plane estimations by adjusting the configuration parameter to obtain the second count.

In some embodiments, the obtaining a plurality of receiving plane estimations corresponding to each of the one or more receiving planes includes determining a count of the plurality of receiving plane estimations corresponding to the each of the one or more receiving planes based on a user input parameter and determining the plurality of receiving plane estimations corresponding to the each of the plurality of receiving planes based on the count of the plurality of receiving plane estimations.

In some embodiments, the generating a plurality of ultrasonic echo signal estimations each of which corresponds to one of the plurality of receiving plane estimations includes processing one or more ultrasonic echo signals corresponding to one of the receiving planes using a beamforming algorithm to obtain the plurality of ultrasonic echo signal estimations of the ultrasonic echo signal corresponding to the plurality of receiving plane estimations.

In some embodiments, the generating a two-dimensional ultrasound image of each of the receiving plane estimations based on the plurality of ultrasonic echo signal estimations includes in response to an overlap of receiving plane estimations corresponding to different receiving planes among the one or more receiving planes, obtaining ultrasonic echo signal estimations corresponding to the receiving plane estimations at an overlapping position of the receiving plane estimations, and performing superposition processing on the ultrasonic echo signal estimations at the overlapping position using a superposition algorithm to obtain a superposed ultrasonic echo signal estimation; generating a two-dimensional ultrasound image corresponding to the overlapping position based on the superposed ultrasonic echo signal estimation.

In some embodiments, the performing superposition processing on the ultrasonic echo signal estimations at the overlapping position using a superposition algorithm to obtain a superposed ultrasonic echo signal estimation includes performing a coherent superposition processing or an incoherent superposition processing on the ultrasonic echo signal estimations at the overlap position based on weight values corresponding to different ultrasonic echo signal estimations at the overlapping position to obtain the superposed ultrasonic echo signal estimation. The weight values corresponding to the ultrasonic echo signal estimations are obtained based on an included angle between the receiving plane estimations at the overlapping position and the corresponding receiving planes corresponding to the receiving plane estimations at the overlapping position.

In some embodiments, the generating a two-dimensional ultrasound image of each of the plurality of receiving plane estimations based on the plurality of ultrasonic echo signal estimations includes in response to determining that a target receiving plane estimation does not overlap with other receiving plane estimations among the plurality of receiving plane estimations, obtaining each of target ultrasonic echo signal estimations corresponding to the target receiving plane estimation, and generating the two-dimensional ultrasound image of each of the target receiving plane estimations based on the target ultrasonic echo signal estimations.

In some embodiments, before obtaining a three-dimensional ultrasound image of the subject based on two-dimensional ultrasound images corresponding to the one or more receiving planes, the method further includes in response to determining that receiving plane estimations corresponding to different receiving planes among the one or more receiving planes overlaps, obtaining two-dimensional ultrasound images corresponding to the receiving plane estimations at an overlapping position of the receiving plane estimations, and performing a superposition processing on the two-dimensional ultrasound images at the overlapping position using an incoherent superposition algorithm to obtain an updated two-dimensional ultrasound image corresponding to the overlapping position.

In some embodiments, the obtaining a three-dimensional ultrasound image of the subject based on the two-dimensional ultrasound images includes processing the two-dimensional ultrasound images using a filtering algorithm and a three-dimensional interpolation algorithm to generate the three-dimensional ultrasound image of the subject.

In some embodiments, the three-dimensional interpolation algorithm includes two or more interpolation processes.

In some embodiments, the method further includes obtaining the three-dimensional ultrasound image of the subject at different acquisition times to generate a four-dimensional ultrasound image of the subject.

An aspect of the present disclosure provide also provides an ultrasound image generation system. The system includes at least one storage device, comprising an instruction set. The system includes at least one processor in communication with the at least one storage device. When the instruction set is executed, the at least one processor is configured to obtain a plurality of ultrasonic echo signals of a subject. The plurality of ultrasonic echo signals are acquired based on one or more receiving planes corresponding to one or more transmitting planes. The at least one processor is configured to obtain a plurality of receiving plane estimations corresponding to the one or more receiving planes and generate a plurality of ultrasonic echo signal estimations each of which corresponds to one of the plurality of receiving plane estimations based on the plurality of ultrasonic echo signals. The at least one processor is configured to generate a two-dimensional ultrasound image of each of plurality of receiving plane estimations based on the plurality of ultrasonic echo signal estimations corresponding to the plurality of receiving plane estimations. The at least one processor is also configured to obtain a three-dimensional ultrasound image of the subject based on two-dimensional ultrasound images of the plurality of receiving plane estimations.

In some embodiments, the obtaining a plurality of receiving plane estimations corresponding to the receiving planes includes the plurality of receiving plane estimations corresponding to each of the receiving planes based on a configuration parameter of an ultrasound device.

An aspect of the present disclosure provides an ultrasound image generation apparatus.

The apparatus includes a processor. The processor is configured to execute the ultrasound image generation method.

An aspect of the present disclosure provides a computer-readable storage medium. The storage medium stores a computer instruction. When a computer reads the computer instructions from the computer-readable storage medium, the computer executes the ultrasound image generation method.

An aspect of the present disclosure provides an ultrasound image generation method. The method includes obtaining a plurality of sets of ultrasonic echo signals of a subject, each set of the ultrasonic echo signals being acquired via one transmission pulse. The method includes, for each of at least one set of the plurality of sets of ultrasonic echo signals, generating a plurality of sets of ultrasonic echo signal estimations based on the each of at least one set of the plurality of sets of ultrasonic echo signals. The method includes determining a plurality of 2D images based on the plurality of sets of ultrasonic echo signal estimations for the each of at least one set of the plurality of sets of ultrasonic echo signals. The method also includes determining a 3D image based on the plurality of 2D images.

In some embodiments, the generating a plurality of sets of ultrasonic echo signal estimations based on the each of at least one set of the plurality of sets of ultrasonic echo signals includes processing each of the plurality of sets of ultrasonic echo signals using a beamforming algorithm to obtain the plurality of sets of ultrasonic echo signal estimations corresponding to each of the plurality of sets of ultrasonic echo signals.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a schematic diagram of an application scenario of an ultrasound image generation system according to some embodiments of the present disclosure;

FIG. 2 is a flowchart of an exemplary process for ultrasound image generation according to some embodiments of the present disclosure;

FIG. 3 is a flowchart of an exemplary process for generating a two-dimensional ultrasound image according to some embodiments of the present disclosure;

FIG. 4 is a flowchart of an exemplary process for obtaining an updated two-dimensional ultrasound image according to some other embodiments of the present disclosure;

FIG. 5 is a first schematic diagram of a transmitting plane and a receiving plane according to some embodiments of the present disclosure;

FIG. 6 is a second schematic diagram of a transmitting plane and a receiving plane according to some embodiments of the present disclosure;

FIG. 7 is a flowchart illustrating another exemplary process for ultrasound image generation according to some embodiments of the present disclosure;

FIG. 8 is a schematic diagram of an ultrasound image generation system according to some embodiments of the present disclosure; and

FIG. 9 is a schematic diagram of an electronic device according to some embodiments of the present disclosure.

### DETAILED DESCRIPTION

In order to more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system,” “device,” “unit,” and/or “module” as used herein is a manner used to distinguish different components, elements, parts, sections, or assemblies at different levels. However, if other words serve the same purpose, the words may be replaced by other expressions.

As shown in the present disclosure and claims, the words “one,” “a,” “a kind,” and/or “the” are not especially singular but may include the plural unless the context expressly suggests otherwise. In general, the terms “comprise,” “comprises,” “comprising,” “include,” “includes,” and/or “including” merely prompt to include operations and elements that have been clearly identified, and these operations and elements do not constitute an exclusive listing.

The present disclosure uses flowcharts to illustrate the operations performed by the system according to the embodiments of the present disclosure. It should be understood that the preceding or following operations are not necessarily performed precisely in sequence. Instead, the operations may be processed in reverse order or simultaneously. Meanwhile, other operations may be added to these processes, or one or more operations may be removed from these processes.

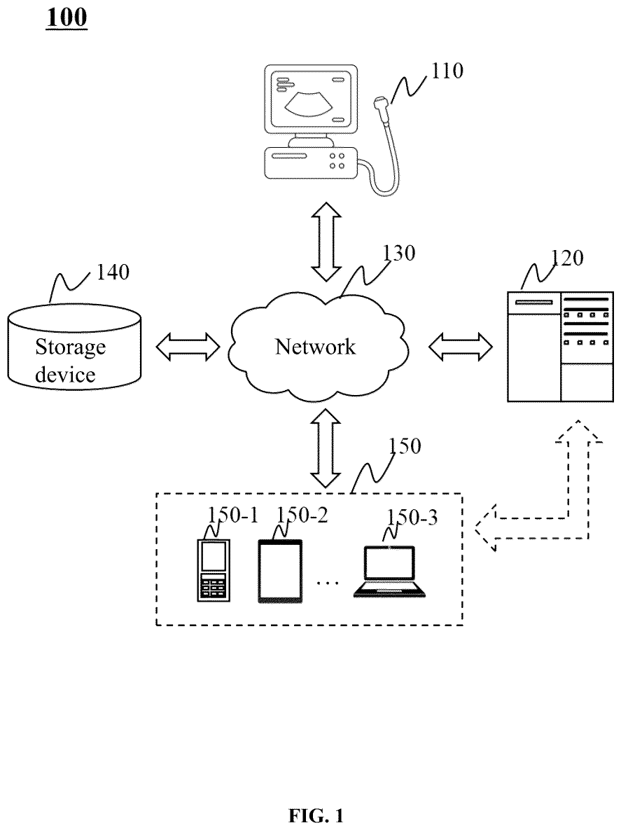

FIG. 1 is a schematic diagram of an application scenario of an ultrasound image generation system according to some embodiments of the present disclosure.

As shown in FIG. 1, an ultrasound image generation system 100 may include an ultrasound device 110, a processing device 120, a network 130, a storage device 140, and a terminal 150. In some embodiments, the ultrasound device 110, the processing device 120, the network 130, the storage device 140, and the terminal 150 may be connected and/or communicate with each other via wired and/or wireless connections.

The ultrasound device 110 is configured to obtain an ultrasonic echo signal of a subject. In some embodiments, the ultrasound device may utilize the physical characteristics of ultrasound and the differences in acoustic properties of the subject to obtain the ultrasonic echo signal. The ultrasonic echo signal may be used to generate an ultrasound image. Merely by way of example, the ultrasound device 110 may include one or more ultrasound probes. Each of the ultrasound probes includes a plurality of ultrasound arrays. Each ultrasound array includes a plurality of elements capable of transmitting and receiving ultrasound waves. The ultrasound probe is configured to transmit ultrasonic waves to the subject. After passing through organs and tissues with different acoustic impedances and different attenuation characteristics, the ultrasonic waves produces different reflections and attenuations, thereby forming ultrasonic echo signals that may be received by the one or more ultrasound probes. The ultrasound device 110 or the processing device 120 may process (e.g., amplify, convert) and/or display the received ultrasonic echo signals, thereby generating an ultrasound image. In some embodiments, the ultrasound device 110 may include a B-mode ultrasound device, a color Doppler ultrasound device, a cardiac color ultrasound device, a three-dimensional color ultrasound device, or the like, or any combination thereof.

In some embodiments, the ultrasound device 110 may send the ultrasonic echo signal to the processing device 120, the storage device 140, and/or the terminal device 150 via the network 130 for further processing. For example, the ultrasonic echo signal obtained by the ultrasound device 110 may be in a non-image form. The non-image form data may be sent to the processing device 120 for generating an ultrasound image.

The processing device 120 may process data and/or information obtained from the ultrasound device 110, the storage device 140, and/or the terminal 150. For example, the processing device 120 may process the ultrasonic echo signal obtained from the ultrasound device 110 and generate an ultrasound image of a target area of a subject. As another example, the processing device 120 may generate a corresponding plurality of receiving planes based on a transmitting plane of the ultrasound device 110. In some embodiments, the ultrasound image may be sent to the terminal 150 and displayed on one or more display devices of the terminal 150. In some embodiments, the processing device 120 may be a single server or a server group. The server group may be centralized or distributed. In some embodiments, the processing device 120 may be local or remote. For example, the processing device 120 may access information and/or data stored in the ultrasound device 110, the storage device 140, and/or the terminal 150 via the network 130. As another example, the processing device 120 may be directly connected to the ultrasound device 110, the storage device 140, and/or the terminal 150 to access the information and/or data stored thereon. As yet another example, the processing device 120 may be integrated into the ultrasound device 110. In some embodiments, the processing device 120 may be implemented on a cloud platform. Merely by way of example, the cloud platform may include a private cloud, a public cloud, a hybrid cloud, a community cloud, a distributed cloud, an internal cloud, a multi-cloud, or the like, or any combination thereof.

In some embodiments, the processing device 120 may be a single processing device that communicates with the ultrasound device 110 and processes data received from the ultrasound device 110.

The network 130 may include any suitable network that may facilitate information and/or data exchange of the ultrasound image generation system 100. In some embodiments, one or more components of the ultrasound image generation system 100 (e.g., the ultrasound device 110, the processing device 120, the storage device 140, or the terminal 150) may be connected and/or communicate with other components of the ultrasound image generation system 100 via the network 130. For example, the processing device 120 may obtain the ultrasonic echo signal from the ultrasound device 110 via the network 130. As another example, the processing device 120 may obtain a user instruction from the terminal 150 via the network 130. The user instruction may be used to instruct the ultrasound device 110 to perform imaging and/or radiation therapy. In some embodiments, the network 130 may include one or more network access points. For example, the network 130 may include wired and/or wireless network access points, such as base stations and/or Internet access points, through which one or more components of the ultrasound image generation system 100 connect to the network 130 to exchange data and/or information.

The storage device 140 may store data and/or instructions. In some embodiments, the storage device 140 may store data obtained from the terminal 150 and/or the processing device 120. In some embodiments, the storage device 140 may store data and/or instructions that the processing device 120 may execute or use to execute the exemplary methods described in the present disclosure. In some embodiments, the storage device 140 may be implemented on a cloud platform. Merely by way of example, the cloud platform may include a private cloud, a public cloud, a hybrid cloud, a community cloud, a distributed cloud, an internal cloud, a multi-cloud, or any combination thereof.

In some embodiments, the storage device 140 may be connected to the network 130 to communicate with one or more components of the ultrasound image generation system 100 (e.g., the processing device 120, the terminal 150, etc.). One or more components of the ultrasound image generation system 100 may access the data or instructions stored in the storage device 140 via the network 130. In some embodiments, the storage device 140 may be directly connected to or communicate with one or more components of the ultrasound image generation system 100 (e.g., the processing device 120, the terminal 150, etc.). In some embodiments, the storage device 140 may be part of the processing device 120.

The terminal 150 may include a mobile device 150-1, a tablet computer 150-2, a laptop computer 150-3, or any combination thereof. In some embodiments, the terminal 150 may remotely operate the ultrasound device 110. In some embodiments, the terminal 150 may operate the ultrasound device 110 via a wireless connection. In some embodiments, the terminal 150 may receive information and/or instructions input by a user, and send the received information and/or instructions to the ultrasound device 110 or the processing device 120 via the network 130. In some embodiments, the terminal 150 may receive data and/or information from the processing device 120. In some embodiments, the terminal 150 may be part of the processing device 120. In some embodiments, the terminal 150 may be omitted.

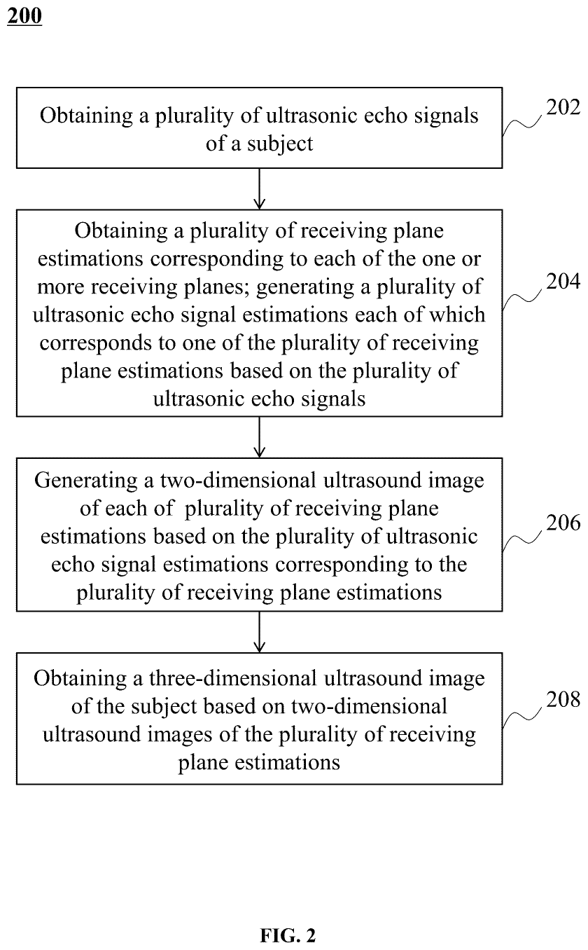

FIG. 2 is a flowchart of an exemplary process for ultrasound image generation according to some embodiments of the present disclosure. As shown in FIG. 2, a process 200 includes the following operations. In some embodiments, the process 200 may be performed by the ultrasound image generation system 100 or a processing device (e.g., the processing device 120). In 202, a plurality of ultrasonic echo signals of a subject may be obtained.

The subject is an object or biological tissue scanned by ultrasound waves generated by an ultrasound device. The subject may be biological or non-biological. For example, the subject includes a patient, a man-made object, or the like. As another example, the subject includes a specific part, organ, tissue, and/or body part of a patient. Merely by way of example, the subject includes the head, the brain, the neck, the body, the shoulders, the arm, the chest, the heart, the stomach, blood vessels, soft tissues, knees, foots, or any combination thereof.

An ultrasonic echo signal refers to an electrical signal generated by the ultrasound device after ultrasound waves is reflected back from the subject and received by an ultrasound probe of the ultrasound device after ultrasound transmission.

In some embodiments, the plurality of ultrasonic echo signals may be acquired based on one or more receiving planes corresponding to one or more transmitting planes. Each of the one or more transmitting planes may correspond to one receiving plane, that is, transmitting plane and receiving plane is one-to-one correspondence. Each of the one or more receiving planes may correspond to one or more ultrasonic echo signals.

A transmitting plane refers to a coverage area formed by transmitting ultrasonic waves along a preset transmitting direction. A transmitting plane may also be referred to as an ultrasonic transmitting plane. The transmitting plane may represent a transmission range of an ultrasonic beam, which is formed by controlling elements in the ultrasound probe. For example, in an ultrasound device, the transmitting plane is a two-dimensional plane whose direction is determined by focusing parameters of a probe array, such as a sector scanning plane.

The receiving plane refers to a coverage area formed by receiving ultrasonic waves reflected via the subject along the preset transmitting direction.

The ultrasound probe includes a plurality of ultrasound arrays. Each ultrasound array includes a plurality of elements capable of transmitting and/or receiving ultrasonic waves. When controlling an ultrasound array to transmit ultrasonic waves outward, each element in the ultrasound array transmits ultrasonic waves along a preset transmission path, forming a transmitting plane with a certain coverage area. The echo signals generated by the ultrasonic waves reflected by the subject return along the preset transmission path and are received by the ultrasound probe, forming an receiving plane with a certain coverage area. The transmitting plane and the receiving plane coincide. Each of the transmitting planes may be formed by one of the plurality of ultrasound arrays in the ultrasound probe. In other words, each of the transmitting planes and the receiving plane coincided with the transmitting plane may correspond to one of the plurality of ultrasound arrays.

In some embodiments, the processing device may control different ultrasound arrays in the ultrasound probe to transmit ultrasonic waves to the subject and receive ultrasonic echo signals reflected back via the subject, thereby obtaining the plurality of ultrasonic echo signals of the subject.

In 204, a plurality of receiving plane estimations corresponding to the one or more receiving planes may be obtained and a plurality of ultrasonic echo signal estimations each of which corresponds to one of the plurality of receiving plane estimations may be generated based on the plurality of ultrasonic echo signals. In some embodiments, as each of the receiving planes may correspond to one of the transmitting planes, and the plurality of receiving plane estimations corresponding to a receiving plane may also correspond to a transmitting plane of the receiving plane.

A receiving plane estimation corresponding to a receiving plane refers to a virtual receiving plane outside the receiving plane. A receiving plane estimation corresponding to a receiving plane is used to expand a data acquisition range and increase an amount of imaging information of the receiving plane. In some embodiments, the receiving plane estimation corresponding to a receiving plane and the receiving plane form a certain included angle. For example, the plurality of receiving plane estimations corresponding to a receiving plane is distributed in a certain manner along an elevation direction of the receiving plane. For example, for a receiving plane, N receiving plane estimations (e.g., N=4) may be generated. The N receiving plane estimations may be located on both sides of the receiving plane. The N receiving plane estimations intersect at the same straight line (e.g., a rotation axis) and spread out to both sides around the rotation axis at specific angular intervals to form a fan-shaped spatial structure. The rotation axis may coincide with an axis of the ultrasound probe. For example, included angles relative to the receiving plane being−1.5°, −0.5°, 0.5°, and 1.5°, respectively. The elevation direction refers to a direction perpendicular to the ultrasound array plane (i.e., transmitting plane) in the ultrasound probe and along which a sector scan or deflection is performed along the long axis of the ultrasound array.

In some embodiments, the processing device may determine the plurality of receiving plane estimations corresponding to one of the receiving planes based on a configuration parameter of the ultrasound device.

The configuration parameter refers to a system parameter adjustable by a user. The configuration parameter may be used to control generation of the plurality of receiving plane estimations. For example, the configuration parameter may be used to control a generation count of the plurality of receiving plane estimations corresponding to one of the receiving planes, an included angle between the each of the plurality of receiving plane estimations and the corresponding receiving plane, etc. For example, the configuration parameter may include an elevation angle spread α (e.g., 3°) and a count N of the plurality of receiving plane estimations. The elevation angle spread represents an included angle between adjacent transmitting planes (or receiving planes).

In some embodiments, to enable the user to flexibly adjust the receiving plane estimations corresponding to the transmitting plane (or the receiving plane) according to different clinical application scenarios, a plurality of different receiving plane estimations corresponding to each of different receiving planes are determined according to the configuration parameter by setting the configuration parameter that is adjustable by the user. For example, the user may adjust a count of the receiving plane estimations corresponding to each transmitting plane, an included angle between each receiving plane estimation and the receiving plane, etc., through the configuration parameter, so as to quickly adapt to different clinical requirements without increasing cost and imaging complexity.

In some embodiments, the processing device may determine a first count of the transmitting planes and a second count of the plurality of receiving plane estimations corresponding to each of the transmitting planes based on the configuration parameter. The plurality of receiving plane estimations corresponding to a receiving plane may include a plurality of different virtual receiving planes sequentially arranged along two sides of the receiving plane.

The first count refers to a total count of transmitting planes used in an ultrasound imaging process (e.g., used for obtaining the plurality of ultrasonic echo signals of the subject in operation 202) of the subject. The first count may be set by setting the configuration parameter. For example, the user may directly set the first count of the transmitting plane in the configuration parameter. In some embodiments, the configuration parameter of the ultrasound device may have a default parameter (e.g., a factory setting or a setting used in a previous scan), such as an initial first count of the transmitting planes. The first count of the transmitting planes may be a count of the transmitting planes obtained after adjusting the initial first count. The initial first count may be a defaulting setting of the system.

The second count refers to a count of the receiving plane estimations corresponding to a transmitting plane or a receiving plane of the transmitting plane. The second count may also be set by setting the configuration parameter. For example, the second count is set to 4, indicating that each transmitting plane or receiving plane of the transmitting plane corresponds to four receiving plane estimations.

A virtual receiving plane corresponding to a receiving plane refer to a receiving plane estimation virtually generated through an algorithm. The virtual receiving planes corresponding to a receiving plane are sequentially arranged along two sides of the receiving plane. The virtual receiving plane corresponding to a receiving plane forms a certain included angle with the receiving plane to simulate a receiving path at different angle. For example, for a certain receiving plane, the virtual receiving planes corresponding to the receiving plane include planes located on both sides of the receiving plane, and included angles between the virtual receiving planes and the receiving plane may be −1.5°, −0.5°, 0.5°, and 1.5°.

In some embodiments, the processing device may determine an included angle between each of the plurality of different virtual receiving planes and the receiving plane based on an elevation angle spread of the receiving plane and the second count of the receiving plane estimations. The elevation angle spread represents an included angle between the receiving plane and an adjacent receiving plane of the receiving plane. The processing device may determine the plurality of the virtual receiving planes arranged along the two sides of the receiving plane based on the included angle between each of the plurality of different virtual receiving planes and the receiving plane.

In some embodiments, the receiving plane estimations corresponding to each receiving plane may be consisted of only different virtual receiving planes. For example, all receiving plane estimations are virtual receiving planes obtained by calculation. In some embodiments, the receiving plane estimations corresponding to each receiving plane may consist of the receiving plane and the plurality of different virtual receiving planes. The virtual receiving planes corresponding to the receiving plane may be generated by deflecting the corresponding receiving plane at different angles (the angle is not 0).

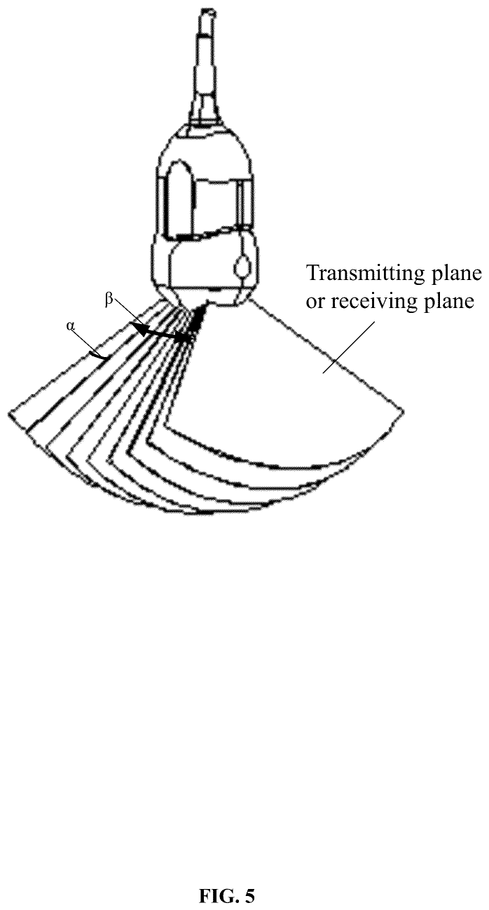

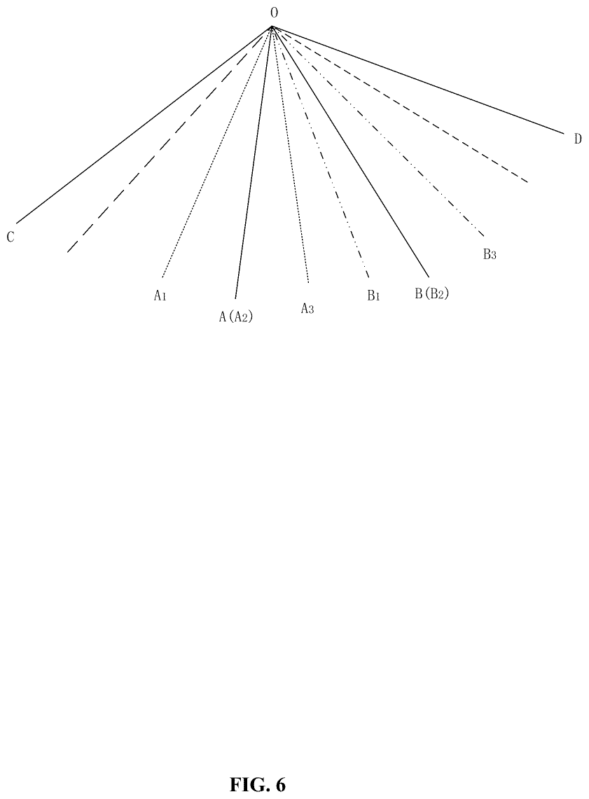

FIG. 5 is a first schematic diagram of a transmitting plane and a receiving plane according to some embodiments of the present disclosure. FIG. 6 is a second schematic diagram of a transmitting plane and a receiving plane according to some embodiments of the present disclosure.

As shown in FIG. 5 to FIGS. 6, A, B, C, and D are four different transmitting planes. A₁, A₂, and A₃ are receiving plane estimations corresponding to the transmitting plane A. B₁, B₂, and B₃ are receiving plane estimations corresponding to the transmitting plane B. Since each of the transmitting planes coincides one of the receiving planes corresponding to the transmitting plane, the receiving plane estimation A₂ is the receiving plane A (i.e., the transmitting plane A) and the receiving plane estimation B₂ is the receiving plane B (i.e., the transmitting plane B). The receiving plane estimations A₁ and A₃ are different virtual receiving planes corresponding to the receiving plane A. The receiving plane estimations B₁ and B₃ are different virtual receiving planes corresponding to the receiving plane B. That is, the receiving plane estimations corresponding to the receiving plane A include the receiving plane A and the virtual receiving planes A₁ and A₃. The receiving plane estimations corresponding to the receiving plane B include the receiving plane B and the virtual receiving planes B₁ and B₃.

The spatial positions of different receiving plane estimations corresponding to each transmitting plane are jointly determined based on the elevation angle spread α of the transmitting plane and the count N of the receiving plane estimations. The elevation angle spread is obtained according to a field of view spread β and a count M of the transmitting planes. M and N are both positive integers. For example, as shown in FIG. 6, an elevation angle spread between transmitting plane C and transmitting plane A, an elevation angle spread between transmitting plane A and transmitting plane B, and an elevation angle spread between transmitting plane B and transmitting plane D are all a, which may be expressed as ∠COA=∠AOB=∠BOD=α.

The field of view spread β of an ultrasound probe determines a field of view range that the ultrasound probe may cover. The field of view spread β is a 3D field of view spread, which represents a spread of an entire 3D image in an elevation direction and is determined by a maximum included angle formed by the transmitting planes. An angle of the elevation angle spread α of each transmitting plane is

$$α = \frac{β}{( M - 1 )} .$$

For example, β=90°, M=21, then α=4.5°.

After the elevation angle spread α of each transmitting plane is obtained, a distribution angle θ of the receiving plane estimations corresponding to each transmitting plane may be obtained according to the elevation angle spread α and the count N of the receiving plane estimations, thereby obtaining an included angle between each receiving plane estimation and the receiving plane. The distribution angle θ refers to an included angle θ between a first receiving plane estimation and a second receiving plane estimation corresponding to the transmitting plane, the first receiving plane estimation and the second receiving plane estimation are respectively a farthest receiving plane estimation to be processed on either side of the transmitting plane. The plurality of different virtual receiving planes are obtained by arranging them sequentially along two sides of the receiving plane according to the calculated included angle between each receiving plane estimation and the receiving plane, so as to obtain the receiving plane estimations corresponding to the receiving plane. As shown in FIG. 6, if ∠A₁OA₃=∠B₁OB₃=θ. θ may be obtained by

$$\frac{α}{N} * 2 or \frac{2 α}{( N + 1 )} * 2 .$$

For example, α=4.5°, N=3, θ=3° is calculated by

$$\frac{α}{N} * 2 , or θ = 4 . {5}^{∘}$$

is calculated by

$$\frac{2 α}{( N + 1 )} * 2 .$$

Since a larger included angle between the receiving plane estimation and the receiving plane leads to a greater deviation between the ultrasonic echo signal estimation calculated based on the ultrasonic echo signal and an actual ultrasonic echo signal, resulting in a less accurate generated two-dimensional ultrasound image, the N receiving plane estimations forming different included angles with the receiving plane may be obtained by arranging them sequentially along two sides of the corresponding receiving plane within the θ range (i.e., an angle between a first receiving plane estimation and a last receiving plane estimation arranged in sequence). For example, taking N=3, θ=3° as an example, a first receiving plane estimation may be set at a position 1.5° to the left of the receiving plane, a second receiving plane estimation may coincide with the receiving plane, and a third receiving plane estimation may be set at a position 1.5° to the right of the receiving plane. If the receiving plane is taken as 0°, then angles of the three receiving plane estimations relative to the receiving plane are −1.5°, 0°, and 1.5°, respectively.

In some embodiments, in response to a volume of the subject being greater than a preset volume, at least one of the first count of the transmitting planes or the second count of the receiving plane estimations is increased by adjusting the configuration parameter For example, the count of the transmitting planes is adjusted from the initial first count to the first count.

A larger volume of the subject corresponds to a larger scanning area. During scanning, a detection frame rate needs to be reduced, so the first count of the corresponding transmitting planes and/or the second count receiving plane estimations increases accordingly. At this time, the user may adjust the configuration parameter, such as increasing a set value of the first count of the transmitting planes and/or increasing a value of the second count of the receiving plane estimations, to increase the first count of the transmitting planes and/or the second count of the receiving plane estimations.

It should be understood that, since the second count of the receiving plane estimations corresponding to each of the transmitting planes has a corresponding relationship with the first count of the transmitting planes, when the user only adjusts the first count of the transmitting planes, the processing device may automatically adjust a total count (a total count of all the receiving plane estimations) of the receiving plane estimations according to the change in the first count.

In some embodiments, in response to a heartbeat frequency of the subject being greater than a preset frequency, at least one of the first count of the transmitting planes or the second count of the receiving plane estimations is reduced by adjusting the configuration parameters.

The heartbeat frequency refers to a periodic motion frequency of the subject. For example, the heartbeat frequency may be a heart beating frequency. A higher heartbeat frequency requires a faster acquisition speed, and the first count of the corresponding transmitting planes and/or the second count of receiving plane estimations decreases accordingly. For example, the count of the transmitting planes and/or the count of the receiving plane estimations may be reduced proportionally. For instance, if a ratio of the count of the transmitting planes to the count of the receiving plane estimations is 1:5, originally there were 3 transmitting planes and 15 receiving plane estimations, which may be proportionally reduced to 2 transmitting planes and 10 receiving plane estimations. That is, while reducing the first count of transmitting planes, the total count of receiving plane estimations is simultaneously reduced.

In this embodiment, the user may adjust the first count of the transmitting plane and the second count of the receiving plane estimations corresponding to each transmitting plane through the configuration parameter to meet different three-dimensional image generation requirements. Taking the subject being the heart as an example, when the volume (shape) of the heart is large, to achieve comprehensive acquisition of the heart, a field of view spread β of the ultrasound probe needs to be increased, and a corresponding count M of the transmitting planes also needs to be increased to ensure imaging quality. Meanwhile, to improve clarity of the three-dimensional ultrasound image and increase details of the three-dimensional ultrasound image, a count N of the receiving plane estimations corresponding to each transmitting plane may be increased. When the heartbeat frequency of the heart is faster, to obtain a four-dimensional ultrasound image of the heart, a generation rate of each three-dimensional ultrasound image needs to be ensured. Therefore, the count M of the transmitting planes may be reduced, and simultaneously a count N of the receiving plane estimations corresponding to each transmitting plane may be reduced, to reduce reconstruction calculation amount of the three-dimensional ultrasound image and increase the generation rate of the three-dimensional ultrasound image.

In some embodiments, the processing device may determine a count of the plurality of receiving plane estimations corresponding to the each of the receiving planes based on a user input parameter; and determining the plurality of receiving plane estimations corresponding to each of the plurality of receiving planes based on the count of the plurality of receiving plane estimations.

The user input parameter refers to a configuration parameter manually input by a user through an interface (e.g., a software interface or a control panel) of the ultrasound device. The user input parameter may be used to customize settings of an ultrasound imaging process. For example, in clinical ultrasound scanning, the user input parameter includes the first count of the transmitting planes, the second count of the receiving plane estimations corresponding to each of the transmitting planes, an included angle between the receiving plane estimation and the transmitting plane, etc.

In some embodiments, the processing device may convert the user input parameter into the configuration parameter of the ultrasound device, and then determine the first count of the transmitting plane and the second count of the receiving plane estimation according to the configuration parameter. A specific determination manner may be the same as the manner described above, and details are not repeated herein.

In some embodiments, the processing device may automatically determine the first count of the transmitting planes and/or the second count of the receiving plane estimations corresponding to each of the transmitting planes based on information of the subject (e.g., a patient), scanning parameters, actual requirements (e.g., requiring higher image resolution or computational efficiency), etc.

For example, the processing device may use a trained machine learning model or an algorithm such as data statistical analysis to determine the first count of transmitting planes and/or the second count of receiving plane estimations corresponding to each of the transmitting planes based on data including a scanning site, a patient body type, a gender, an age, etc., and an actual requirements. Based on the first count of transmitting planes, an elevation angle spread of each receiving plane (i.e., the elevation angle spread of the transmitting plane) may be further determined, thereby determining an included angle of each of the receiving plane estimations corresponding to each receiving plane and the receiving plane in combination with the second count, to obtain a corresponding plurality of receiving plane estimations.

In some embodiments, when higher efficiency imaging is required, the first count and/or the second count determined by the processing device may be relatively small, corresponding to a reduced data processing amount and improved efficiency. When imaging with higher image quality requirements is needed, the second count determined by the processing device may be larger, and the determined receiving plane estimations corresponding to a receiving plane may include the receiving plane (the ultrasonic echo signal corresponding to the receiving plane does not require additional processing, which can avoid errors introduced during the calculation process, making the finally included information relatively more accurate). When both imaging efficiency and imaging image quality are required, the processing device may determine an optimal second count that may achieve a balance. For example, a trained machine learning model may be used for determining the first count and/or the second count. The input of the trained machine learning model includes patient information and scanning parameters. The output of the trained machine learning model includes the second count. Alternatively, the output of the trained machine learning model includes the first count and the second count. As another example, the input of the trained machine learning model includes patient information, scanning parameters, and the first count, and the outputs of the trained machine learning model includes the second count.

Training data for the machine learning model includes sample patient information, sample scanning parameters, a sample first count, and a sample second count. The model may include Linear Regression, Neural Networks, etc. The second count and/or the first count may serve as model labels.

In some embodiments, the first count and the second count may be determined before scanning, or may be determined through real-time updating during the scanning process. For example, the system or the user may evaluate a quality of a two-dimensional ultrasound image or a three-dimensional ultrasound image obtained based on an initial second count and an initial first count. The initial second count and the initial first count may be default setting of the system. In response to a determination that a preset condition is not satisfied (e.g., being less than a preset resolution threshold), the second count and/or the first count may be recalculated to obtain an updated second count and/or an updated first count. The ultrasound image is obtained again based on the updated second count and/or the updated first count.

In some embodiments, the processing device may further determine the first count and/or the second count according to a system performance. For example, a higher CPU processing capability of the system allows for determining a larger value of the second count (a greater count of receiving plane estimations results in better image quality). A weaker processing capability results in determining a smaller value of the second count. The processing device may determine whether the receiving plane estimations corresponding to a receiving plane include the receiving plane based on the system performance, the scanning site, and a requirement for image quality.

In some embodiments, virtual receiving planes on the left and right sides of the receiving plane are symmetric (e.g., as shown in FIG. 6, the virtual receiving plane A1 on the left side and the virtual receiving plane A3 on the right side of the original receiving plane A are symmetric). For example, included angles between the receiving plane and each of the virtual receiving planes corresponding to the receiving plane are identical. Alternatively, the included angles between the receiving plane and each of the respective virtual receiving planes corresponding to the receiving plane may be set to be asymmetric. For example, included angles between the receiving plane and each of at least some of the virtual receiving planes corresponding to the receiving plane are different.

After determining the different receiving plane estimations corresponding to each receiving plane, a delay calculation of the ultrasonic echo signals at different deflection angles is performed via a beamforming algorithm, thereby obtaining the ultrasonic echo signal estimation corresponding to each receiving plane estimation. The delay calculation may calculate an offset of a signal on a time axis based on a geometric relationship and a speed of sound. The deflection angle is an angle of the virtual receiving plane relative to the receiving plane. The receiving plane may have a deflection angle of 0° for reference.

In some embodiments, the processing device may process one or more ultrasonic echo signals corresponding to one of the receiving planes using the beamforming algorithm to obtain the plurality of ultrasonic echo signal estimations of the ultrasonic echo signal corresponding to the plurality of receiving plane estimations corresponding to the one of the receiving planes.

Because distances from a same point of the subject to different elements of an ultrasound array are different, ultrasonic echo signals from the same point on the subject output by the different elements have delay differences. Therefore, to achieve rapid generation of the 3D ultrasound image, reduce a calculation amount, and simultaneously ensure an imaging quality of the 3D ultrasound image, the processing device may perform delay calculation processing for different receiving angles (i.e., the different deflection angles) on the received ultrasonic echo signal via a software algorithm, to obtain the plurality of ultrasonic echo signal estimations corresponding to the plurality of receiving plane estimations. Different receiving angles correspond to different receiving plane estimations. Different receiving plane estimations corresponding to the different receiving angles are obtained by deflecting the receiving plane at different angles (i.e., deflection angles for the different receiving plane estimations). For one receiving plane, deflecting at a same angle toward both sides of the receiving plane also results in different receiving plane estimations.

Thus, after transmitting one ultrasonic pulse, the plurality of ultrasonic echo signal estimations at the plurality of different deflection angles may be obtained. Without requiring additional transmitting planes, one transmitting plane corresponds to a plurality of receiving plane estimations, thereby enabling collection and synthesis of ultrasonic echo signals from the plurality of receiving plane estimations and increasing data richness.

It should be noted that when a deflection angle between the receiving plane estimation and the receiving plane is 0, the ultrasonic echo signal estimation of the receiving plane estimation is the ultrasonic echo signal corresponding to the receiving plane.

It should be noted that the execution order of operation 202 and operation 204 is not limited in the embodiments of the present disclosure. For example, operation 202 may be performed before operation 204. As another example, operation 202 may be performed after operation 204. For instance, the plurality of ultrasonic echo signals of the subject may be obtained after obtaining the plurality of receiving plane estimations corresponding to one or more receiving planes. As a further example, operation 202 and operation 204 may be performed simultaneously. In some embodiments, after a count of the transmitting plane/a count of the receiving plane is obtained/determined, the receiving plane estimations and the ultrasonic echo signals of the subject may be obtained/determined.

In 206, a 2D ultrasound image of each of the plurality of receiving plane estimations may be generated based on the plurality of ultrasonic echo signal estimations corresponding to the plurality of receiving plane estimations.

In some embodiments, after obtaining the plurality of ultrasonic echo signal estimations, the processing device may perform conversion, amplification, filtering, etc., on each ultrasonic echo signal estimation to obtain a processed ultrasonic echo signal, and reconstruct a corresponding 2D ultrasound image based on the processed ultrasonic echo signal. For example, an image reconstruction algorithm may be used to reconstruct the two-dimensional ultrasound image. The present embodiment does not limit the specific reconstruction algorithm. An ultrasonic echo signal estimation corresponding to each receiving plane estimation may be used to generate a two-dimensional ultrasound image.

In some embodiments, the plurality of receiving plane estimations corresponding to different transmitting planes (i.e., receiving planes) may overlap. For example, the transmitting planes may include a first transmitting plane and a second transmitting plane. The first transmitting plane may correspond to a first receiving plane estimation and the second transmitting plane may correspond to a second receiving plane estimation. The first receiving plane estimation and the second receiving plane estimation may overlap. The overlapping refers to that a position of the first receiving plane estimation and a position of the second receiving plane estimation in space are the same. The receiving plane estimations at the overlapping position may be the same or different. For example, for the first transmitting plane corresponding to the first receiving plane estimation and the second transmitting plane corresponding to the second receiving plane estimation, ultrasound arrays corresponding to the first transmitting plane and the second transmitting plane may be the same or different. If the ultrasound arrays are the same, the first receiving plane estimation and the second receiving plane estimation at the overlapping position may be the same. If the ultrasound arrays are different, the first receiving plane estimation and the second receiving plane estimation at the overlapping position may not be the same. It should be noted that, in some embodiments, even if the ultrasound arrays corresponding to the first transmitting plane and the second transmitting plane are different, since the receiving plane estimations are generated virtual receiving planes, the first receiving plane estimation and the second receiving plane estimation at the overlapping position may also be the same.

The overlapping receiving plane estimations may correspond to two ultrasonic echo signal estimations, i.e., the first receiving plane estimation corresponds to a first ultrasonic echo signal estimation and a second receiving plane estimation corresponding to a second ultrasonic echo signal estimation.

For overlapping receiving plane estimations and a non-overlapping receiving plane estimation, the processing device may use different processing manners to generate the corresponding two-dimensional ultrasound image of the receiving plane estimation based on the ultrasonic echo signal estimation.

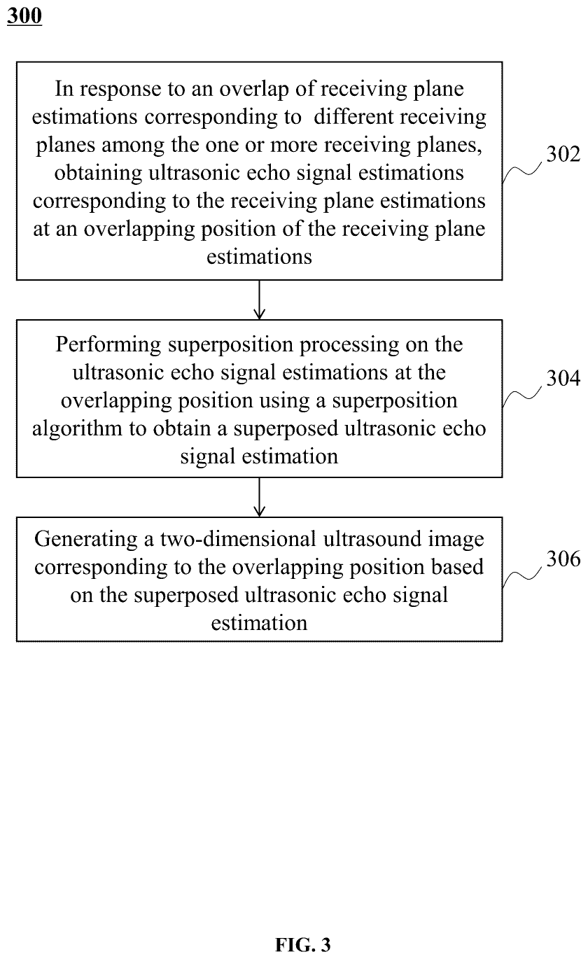

In some embodiments, for overlapping receiving plane estimations, the processing device may use a preset superposition algorithm to perform superposition processing on the ultrasonic echo signal estimations corresponding to the overlapping receiving plane estimations (e.g., the first ultrasonic echo signal estimation and the second ultrasonic echo signal estimation), to obtain a superposed ultrasonic echo signal estimation, and generate the two-dimensional ultrasound image corresponding to the overlapping receiving plane estimations based on the superposed ultrasonic echo signal estimation. More descriptions may be found in the detailed description of FIG. 3.

In some embodiments, in response to determining that a target receiving plane estimation does not overlap with other receiving plane estimations among the plurality of receiving plane estimations, a target ultrasonic echo signal estimation corresponding to the target receiving plane estimation is obtained, and the two-dimensional ultrasound image of the target receiving plane estimation is generated based on the target ultrasonic echo signal estimation. The target receiving plane estimation refers to a receiving plane estimation being processed.

The target receiving plane estimation is a receiving plane estimation that is being processed and does not overlap with the receiving plane estimations corresponding to the transmitting plane. When the target receiving plane estimation does not overlap with other receiving plane estimations, the corresponding two-dimensional ultrasound image is generated directly based on the ultrasonic echo signal estimation corresponding to the target receiving plane estimation.

In 208, a three-dimensional ultrasound image of the subject may be obtained based on two-dimensional ultrasound images of the plurality of receiving plane estimations

In some embodiments, the processing device may reconstruct the 3D image of subject based on the two-dimensional ultrasound images of the plurality of receiving plane estimations corresponding to a plurality of transmitting planes. For example, three-dimensional scan conversion and an interpolation process are performed on the two-dimensional ultrasound images of the plurality of receiving plane estimations to quickly obtain the 3D ultrasound image of the subject.

To eliminate noise and improve data fidelity, and to obtain a high-quality 3D ultrasound image, the processing device may use a 2D filtering algorithm to perform filtering processing on the 2D ultrasound images, and use a 3D filtering algorithm and a three-dimensional interpolation algorithm to convert the plurality of two-dimensional ultrasound images from the different orientations, to generate the high-resolution three-dimensional ultrasound image of the subject. The 2D filtering algorithm and the 3D filtering algorithm may be any one of a Gaussian filtering algorithm, a median filtering algorithm, a hole filling algorithm, etc. The three-dimensional interpolation algorithm may be any one of Bicubic Interpolation, Trilinear Interpolation, etc.

In some embodiments, the three-dimensional interpolation process may include two or more interpolation operations. During the three-dimensional interpolation process, because a first interpolation result obtained after the first interpolation operation may have an issue of alternating brightness and darkness on an interpolation surface, a second interpolation operation may be performed to obtain a higher quality three-dimensional ultrasound image. That is, interpolation is performed again based on the interpolated data and interpolation neighbor data (which may be data obtained through interpolation, or may be original data). The entire 3D interpolation process may involve two or more interpolation operations.

In some embodiments, the processing device may determine whether an interpolation result after the second interpolation satisfies a condition (e.g., whether an image quality satisfies a preset threshold). In response to a determination that the condition is not satisfied, the interpolation process is performed again until the image quality meets the requirement.

In some embodiments, to further accelerate the three-dimensional interpolation and scan conversion for the three-dimensional ultrasound image, the processing device may utilize a parallel processing capability of a GPU to parallelize the acquisition of the ultrasonic echo signal estimations and the generation of the ultrasound image. The scan conversion refers to a process of converting data based on a polar coordinate system collected by an ultrasound probe into a visible image based on a Cartesian coordinate system (rectangular coordinate system).

In some embodiments, the processing device may obtain the three-dimensional ultrasound images of the subject at different acquisition times to generate a 4D ultrasound image of the subject

The four-dimensional ultrasound image refers to a time-series image composed of a sequence of three-dimensional ultrasound images at the different acquisition times, displaying dynamic three-dimensional changes of the subject. The four-dimensional ultrasound image may be obtained by adding a time dimension to the three-dimensional ultrasound image. For example, the plurality of three-dimensional ultrasound images are sorted chronologically according to acquisition time, and the four-dimensional ultrasound image is generated by dynamically displaying the plurality of three-dimensional ultrasound images.

Specifically, by cyclically executing operation 202 to operation 208, the three-dimensional ultrasound images at the different acquisition times may be obtained, thereby generating the four-dimensional ultrasound image of the subject.

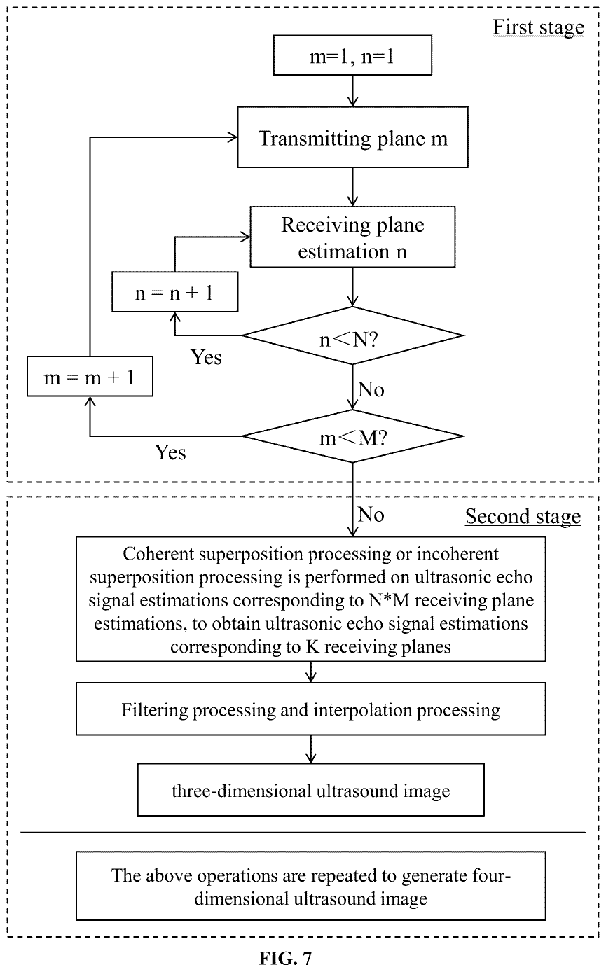

FIG. 7 is a flowchart illustrating another exemplary process for ultrasound image generation according to some embodiments of the present disclosure.

Merely by way of example, as shown in FIG. 7, a first count of transmitting planes is determined as M according to a configuration parameter, and a second count of receiving plane estimations corresponding to each transmitting plane is determined as N. In a first stage, starting from a first receiving plane estimation (n=1) of a first transmitting plane (m=1), the ultrasonic echo signal estimation corresponding to each receiving plane estimation corresponding to the first transmitting plane is sequentially acquired via n=n+1 until n=N, whether the ultrasonic echo signal estimations corresponding to all receiving plane estimations corresponding to the first transmitting plane have been acquired may be determined. The ultrasonic echo signal estimation corresponding to each receiving plane estimation corresponding to a second transmitting plane is acquired via m=m+1, ultrasonic echo signal estimations corresponding to each receiving plane estimation corresponding to all transmitting planes have been acquired until m=M. In a second stage, the coherent superposition processing or the incoherent superposition processing is performed on the ultrasonic echo signal estimations corresponding to N*M receiving plane estimations, to obtain ultrasonic echo signal estimations corresponding to K receiving plane estimations. Filtering processing and interpolation processing are performed on the ultrasonic echo signal estimations corresponding to the K receiving plane estimations to generate the three-dimensional ultrasound image. The above operations are repeated to acquire the three-dimensional ultrasound images at the different acquisition times, thereby generating the four-dimensional ultrasound image.

The first stage and the second stage may be processed in parallel, ensuring seamless connection and high-speed execution of each operation, to rapidly and continuously generate the three-dimensional ultrasound image, obtaining a real-time four-dimensional ultrasound image.

According to some embodiments of the present disclosure, by performing beamforming calculation outside the receiving planes, the two-dimensional ultrasound image corresponding to each transmitting plane under the plurality of receiving planes at different angles is obtained to generate the three-dimensional ultrasound image. This not only increases effective information of the three-dimensional ultrasound image, significantly improving image clarity and detail, but also, compared with existing 3D reconstruction algorithms (without increasing transmitting planes), reduces the count of transmitting planes under the condition of the same receiving planes, thereby reducing the calculation amount during the entire reconstruction process of the three-dimensional ultrasound image and improving an imaging rate. Thus, the four-dimensional ultrasound image is obtained based on the rapid and continuous three-dimensional ultrasound images. Furthermore, adjustable parameters are provided, enabling users to perform adaptive configuration according to different scenarios, quickly adapt to different clinical needs, and provide doctors with more valuable image information.

FIG. 3 is a flowchart of an exemplary process for generating a two-dimensional ultrasound image according to some embodiments of the present disclosure. As shown in FIG. 3, a process 300 includes the following operations. In some embodiments, the process 300 may be performed by the ultrasound image generation system 100 or a processing device (e.g., the processing device 120).

In 302, in response to an overlap of receiving plane estimations corresponding to different receiving planes among the one or more receiving planes, ultrasonic echo signal estimations corresponding to the receiving plane estimations at an overlapping position of the receiving plane estimations may be obtained.

The receiving plane estimations overlapping refers to that receiving plane estimations of different transmitting planes are in the same position in space. The overlapping position is a spatial position of the overlapping receiving plane estimations. There may be two or more overlapping receiving plane estimations at the same spatial position.

In some embodiments, to optimize image quality (e.g., to improve image contrast and clarity), an overlap may be provided between different receiving planes. When the receiving plane estimations corresponding to different receiving planes overlap, the processing device may perform the superposition processing on the ultrasonic echo signal estimations corresponding to the overlapping receiving plane estimations using a preset superposition algorithm to obtain a superposed ultrasonic echo signal estimation corresponding to the overlapping position, and then generate a corresponding two-dimensional ultrasound image based on the superposed ultrasonic echo signal estimation.

For example, the transmitting planes may include a first transmitting plane and a second transmitting plane. The first transmitting plane may correspond to a first receiving plane estimation and the second transmitting plane may correspond to a second receiving plane estimation. The first receiving plane estimation and the second receiving plane estimation may overlap. In other words, the first receiving plane estimation and the second receiving plane estimation may be the same receiving plane estimation. The first receiving plane estimation and the second receiving plane estimation may be two overlapping receiving plane estimations. The first receiving plane estimation corresponds to a first ultrasonic echo signal estimation and a second receiving plane estimation corresponding to a second ultrasonic echo signal estimation. As the overlapping first receiving plane estimation and the second receiving plane estimation correspond to the same receiving plane estimation, the overlapping receiving plane estimations may correspond to two ultrasonic echo signal estimations, i.e., the first ultrasonic echo signal estimation and the second ultrasonic echo signal estimation.

A superposed ultrasonic echo signal estimation corresponding to the overlapping receiving plane estimations may be generated based on the first ultrasonic echo signal estimation and the second ultrasonic echo signal estimation. An ultrasound image corresponding to the overlapping receiving plane estimations may be generated based on the superposed ultrasonic echo signal estimation.

As a further example, referring back to FIG. 6, a position of the transmitting plane A is 0°, and a position of the transmitting plane B is 4.5°. When θ=4.5° and N=3, positions of three receiving plane estimations A₁-A₃ corresponding to the transmitting plane A are −2.25°, 0°, and 2.25°, respectively, and positions of three receiving plane estimations B₁-B₃ corresponding to the transmitting plane B are 2.25°, 4.5°, and 6.25°, respectively. Therefore, for the position of 2.25°, overlapping receiving plane estimations A₃ and B₁ exist. Accordingly, superposition processing may be performed on the ultrasonic echo signal estimation corresponding to A₃ and the ultrasonic echo signal estimation corresponding to B₁ to obtain a superposed ultrasonic echo signal estimation corresponding to the position of 2.25°, and a two-dimensional ultrasound image at the position of 2.25° is generated based on the superposed ultrasonic echo signal estimation. For the positions of −2.25°, 0°, 4.5°, and 6.25°, no overlapping receiving plane estimations exist. Therefore, corresponding two-dimensional ultrasound images are generated based on the ultrasonic echo signal estimations corresponding to A₁, A₂, B₂, and B₃, respectively. Thus, five two-dimensional ultrasound images with different orientations may be obtained through two transmitting planes, providing more image details for generating a three-dimensional ultrasound image.

The ultrasonic echo signal estimation corresponding to each receiving plane estimation at the overlapping position refers to the ultrasonic echo signal estimations corresponding to the two overlapping receiving plane estimations, respectively, such as the ultrasonic echo signal estimation corresponding to the receiving plane estimation A₃ and the ultrasonic echo signal estimation corresponding to the receiving plane estimation B₁.

In 304, superposition processing may be performed on the ultrasonic echo signal estimations at the overlapping position using a superposition algorithm to obtain a superposed ultrasonic echo signal estimation.

The preset superposition algorithm may be a coherent superposition algorithm or an incoherent superposition algorithm (e.g., STB (Synthetic Transmit Beam)). A user may select based on actual requirements for the ultrasound image.

Taking the STB algorithm as an example, for M transmitting planes, and each transmitting plane corresponds to N receiving plane estimations, when the STB algorithm is used, K receiving planes may be finally obtained,

$$K = ( \frac{( N + 1 ) * ( M - 1 )}{2} ) + 1 .$$

In this embodiment, flexible invocation of the coherent superposition algorithm and the incoherent superposition algorithm may significantly improve image clarity and detail.

In some embodiments, the processing device may perform the coherent superposition processing or the incoherent superposition processing on the ultrasonic echo signal estimations at the overlap position based on weight values corresponding to different ultrasonic echo signal estimations at the overlapping position to obtain the superposed ultrasonic echo signal estimation. A weight value corresponding to an ultrasonic echo signal estimation may be obtained based on a size of an included angle between the receiving plane estimation corresponding to the ultrasonic echo signal estimation and the receiving plane corresponding to the receiving plane estimation at the overlapping position.

Considering that a larger angle between the receiving plane estimation and the receiving plane leads to a greater deviation between the ultrasonic echo signal estimation calculated based on the ultrasonic echo signal and an actual ultrasonic echo signal, to further improve image contrast and clarity, a weight value may be set according to an included angle between the receiving plane estimation corresponding to the ultrasonic echo signal estimation and the corresponding receiving plane. A smaller included angle between receiving plane estimation corresponding to the ultrasonic echo signal estimation and the corresponding receiving plane results in a larger set weight value. Conversely, a larger included angle results in a smaller set weight value. Then, superposition processing is performed on the different ultrasonic echo signal estimations based on the weight values corresponding to the different ultrasonic echo signal estimations to obtain the superposed ultrasonic echo signal estimation.

For example, a position of a first transmitting plane is 0°, a position of a second transmitting plane is 2°, positions of four receiving plane estimations A₁-A₄ corresponding to the first transmitting plane are −1.5°, −0.5°, 0.5°, and 1.5°, respectively, and positions of four receiving plane estimations B₁-B₄ corresponding to the second transmitting plane are 0.5°, 1.5°, 2.5°, and 3.5°, respectively. That is, the receiving plane estimations corresponding to the first transmitting plane and the second transmitting plane only include virtual receiving planes A₁-A₄ and B₁-B₄, and do not include the first receiving plane and the second receiving plane. Then, for the position of 0.5°, overlapping receiving plane estimations A₃ and B₁ exist. Since an included angle between A₃ and the first transmitting plane is 0.5°, and an included angle between B₁ and the second transmitting plane is 1.5°, a weight value of the ultrasonic echo signal estimation of A₃ may be set to be large, and a weight value of the ultrasonic echo signal estimation of B₁ may be set to be small. Similarly, for the position of 1.5°, overlapping receiving plane estimations A₄ and B₂ exist, a weight value of the ultrasonic echo signal estimation of A₄ may be set to be small, and a weight value of the ultrasonic echo signal estimation of B₂ may be set to be large.

Certainly, uniform superposition of the different ultrasonic echo signal estimations at the overlapping position may also be set, which is not limited in this embodiment.

In some embodiments, the processing device may also automatically determine whether a weight value needs to be set and a size of a corresponding weight value based on actual situations. For example, the processing device may determine a requirement level for image contrast and clarity based on a scanning site and/or a lesion type, and determine whether to use a weight value based on the requirement level. For example, for tissues such as the abdomen, adipose tissue, bone, peripheral blood vessels, joints, superficial masses, or skin lesions, the requirements for image contrast and clarity are relatively low, and no weight value may be set, i.e., the different ultrasonic echo signal estimations are uniformly superposed. As another example, although the quality requirement for bone is low, when a high-quality image is determined to be needed according to the lesion type of the bone (e.g., when evaluating subtle fractures), a weight value may be set.

In 306, a two-dimensional ultrasound image corresponding to the overlapping position may be generated based on the superposed ultrasonic echo signal estimation.

A manner of generating the two-dimensional ultrasound image based on the superposed ultrasonic echo signal estimation may be the same as the manner of generating the two-dimensional ultrasound image described above, which is not repeated here.

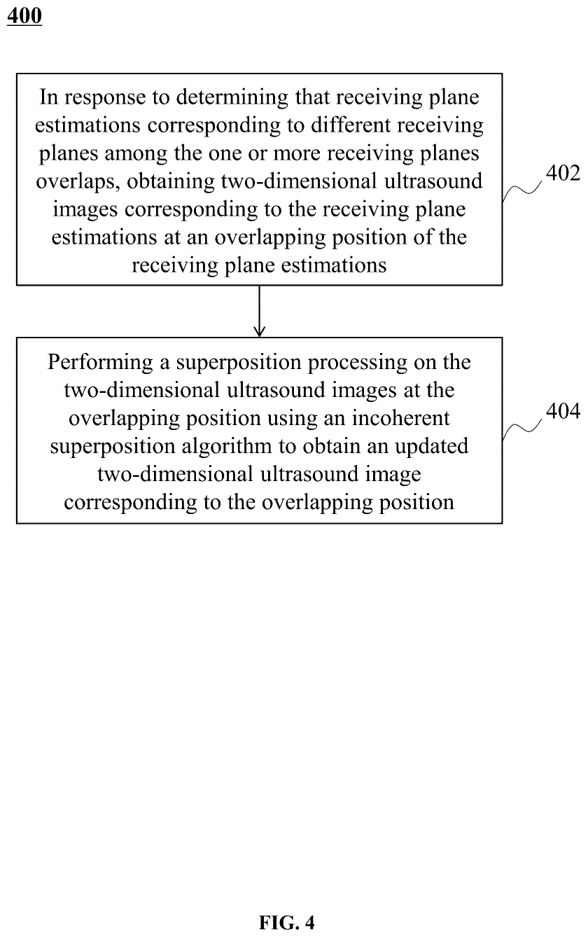

In some embodiments, for the receiving plane estimation at the overlapping position, before determining the three-dimensional ultrasound image of the subject based on the two-dimensional ultrasound image corresponding to each receiving plane estimation, the processing device may also obtain an updated two-dimensional ultrasound image at the overlapping position using a process 400 shown in FIG. 4.

In some embodiments, whether to perform superposition of the ultrasonic echo signals may be automatically determined based on system CPU processing capability, requirements for imaging efficiency, and requirements for image quality. For example, when the CPU processing capability is weak, the requirement for imaging efficiency is high, and the requirement for image quality is low, the corresponding two-dimensional ultrasound image may be directly generated based on the ultrasonic echo signal estimation corresponding to each receiving plane estimation. Conversely, superposition processing is performed on the ultrasonic echo signal estimations at the overlapping position using the preset superposition algorithm to obtain the superposed ultrasonic echo signal estimation, and the two-dimensional ultrasound image corresponding to the overlapping position is generated based on the superposed ultrasonic echo signal estimation.

FIG. 4 is a flowchart of an exemplary process for obtaining an updated two-dimensional ultrasound image according to some other embodiments of the present disclosure. As shown in FIG. 4, a process 400 includes the following operations. In some embodiments, the process 400 may be performed by the ultrasound image generation system 100 or a processing device (e.g., the processing device 120).

In 402, in response to determining that receiving plane estimations corresponding to different receiving planes among the one or more receiving planes overlaps, two-dimensional ultrasound images corresponding to the receiving plane estimations at an overlapping position of the receiving plane estimations may be obtained.

In some embodiments, the processing device may not consider whether overlapping receiving plane estimations exist, and may not perform superposition processing on the ultrasonic echo signal estimations corresponding to the overlapping receiving plane estimations. Instead, the corresponding two-dimensional ultrasound image is directly generated based on the ultrasonic echo signal estimation corresponding to each receiving plane estimation to obtain a plurality of two-dimensional ultrasound images with different orientations.

In 404, a superposition processing may be performed on the two-dimensional ultrasound images at the overlapping position using an incoherent superposition algorithm to obtain an updated two-dimensional ultrasound image corresponding to the overlapping position.

Then, before generating the three-dimensional ultrasound image based on the two-dimensional ultrasound images, perform incoherent superposition processing on the two-dimensional ultrasound images corresponding to the different receiving plane estimations at the overlapping position to obtain the updated two-dimensional ultrasound image corresponding to the overlapping position. The two-dimensional ultrasound images corresponding to the receiving plane estimations without overlap do not need to be updated. Then, the three-dimensional ultrasound image is generated based on the updated two-dimensional ultrasound images and the two-dimensional ultrasound images that do not need to be updated to further improve image contrast and clarity.

Based on the same inventive concept, embodiments of the present disclosure further provide an ultrasound image generation method. The method includes obtaining a plurality of sets of ultrasonic echo signals of a subject, each set of the ultrasonic echo signals is acquired via one transmission pulse. Detailed descriptions may refer to the relevant descriptions of operation 202 in FIG. 2.

The method also includes, for each of at least one set of the plurality of sets of ultrasonic echo signals, generating a plurality of sets of ultrasonic echo signal estimations based on the each of at least one set of the plurality of sets of ultrasonic echo signals. Detailed descriptions may refer to the relevant descriptions of operation 204 in FIG. 2.

The method further includes determining a plurality of 2D images based on the plurality of sets of ultrasonic echo signal estimations for the each of at least one set of the plurality of sets of ultrasonic echo signals. Detailed descriptions may refer to the relevant descriptions of operation 206 in FIG. 2.

In addition, the method includes determining a 3D image based on the plurality of 2D images. Detailed descriptions may refer to the relevant descriptions of operation 208 FIG. 2.

In some embodiments, the generating a plurality of sets of ultrasonic echo signal estimations based on the each of at least one set of the plurality of sets of ultrasonic echo signals includes processing each of the plurality of sets of ultrasonic echo signals using a beamforming algorithm to obtain the plurality of sets of ultrasonic echo signal estimations corresponding to each of the plurality of sets of ultrasonic echo signals. Detailed descriptions may be found in the relevant descriptions of operation 204 in FIG. 2 or FIGS. 5-6 described above.

It should be noted that the descriptions of the various processes above are merely for illustration and explanation, and do not limit the applicable scope of the present disclosure. Those skilled in the art may make various modifications and changes to the processes under the guidance of the present disclosure. However, these modifications and changes still fall within the scope of the present disclosure. For example, adding storage operations between the operations, and adjusting an execution order between some operations, such as the execution order of operation 202 and operation 204 in FIG. 2.

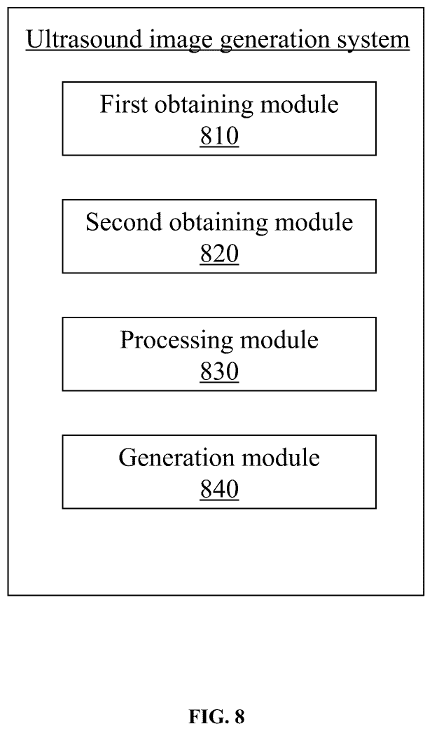

FIG. 8 is a schematic module diagram of an ultrasound image generation system according to some embodiments of the present disclosure. As shown in FIG. 8, the ultrasound image generation system includes a first obtaining module 810, a second obtaining module 820, a processing module 830, and a generation module 840.

The first obtaining module 810 is configured to obtain a plurality of ultrasonic echo signals of a subject. The plurality of ultrasonic echo signals are acquired based on receiving planes corresponding to different transmitting planes. The transmitting plane represents a coverage range formed by transmitting ultrasonic waves along a preset transmitting direction. The receiving plane refers to a coverage area formed by receiving ultrasonic waves reflected via the subject along the preset transmitting direction.

The second obtaining module 820 is configured to obtain a plurality of receiving plane estimations corresponding to each of the receiving planes and generate a plurality of ultrasonic echo signal estimations each of which corresponds to one of the plurality of receiving plane estimations based on the plurality of ultrasonic echo signals.

The processing module 830 is configured to generate a two-dimensional ultrasound image of each of the receiving plan estimations based on the plurality of ultrasonic echo signal estimations corresponding to the plurality of receiving plane estimations.

The generation module 840 is configured to obtain a three-dimensional ultrasound image of the subject based on two-dimensional ultrasound images of the plurality of receiving plane estimations.

For system embodiments, since they basically correspond to method embodiments, relevant parts may refer to the description of the method embodiments. The system embodiments described above are merely illustrative. The units described as separate components may or may not be physically separated, and the components displayed as units may or may not be physical units, that is, they may be located in one place or distributed to multiple network units. Some or all of the modules may be selected according to actual requirements to achieve the purpose of the solution of the present disclosure.

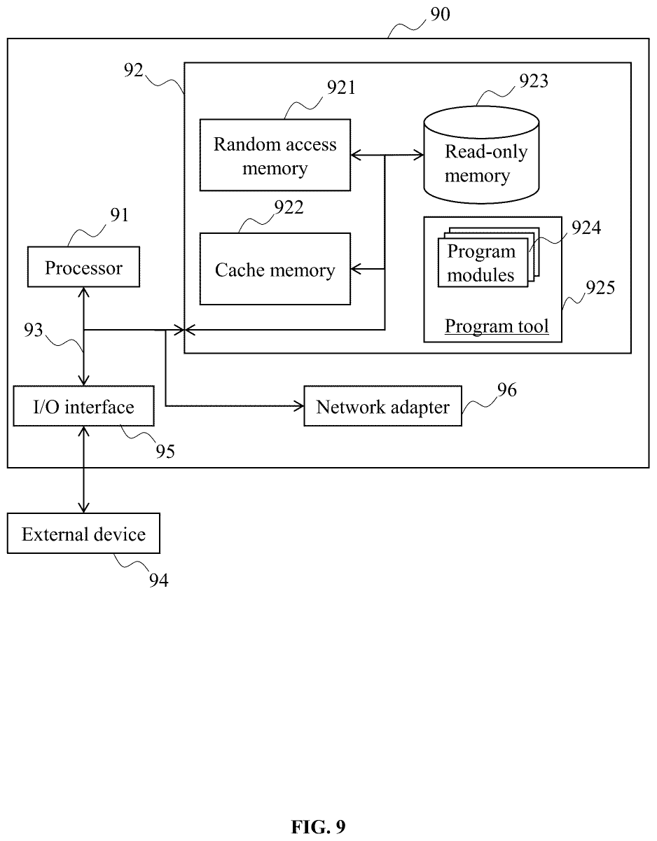

FIG. 9 is a schematic structural diagram of an electronic device according to some embodiments of the present disclosure. The electronic device includes a memory, a processor, and a computer program stored on the memory and executable on the processor. When the processor executes the computer program, the ultrasound image generation method described in any of the above embodiments is implemented. The electronic device 90 shown in FIG. 9 is merely an example and should not impose any limitation on the function and scope of use of the embodiments of the present disclosure.

As shown in FIG. 9, the electronic device 90 may be in the form of a computing device, for example, a server device. Components of the electronic device 90 may include, but are not limited to: the at least one processor 91 described above, the at least one memory 92 described above, and a bus 93 connecting different system components (including the memory 92 and the processor 91).

The bus 93 includes a data bus, an address bus, and a control bus.

The memory 92 may include a volatile memory, such as a random access memory (RAM) 921 and/or a cache memory 922, and may further include a read-only memory (ROM) 923.

The memory 92 may also include a program tool 925 (or utility tool) having a set (at least one) of program modules 924. Such program modules 924 include, but are not limited to: an operating system, one or more application programs, other program modules, and program data. Each or some combination of these examples may include an implementation of a network environment.

The processor 91 executes various functional applications and data processing by running computer programs stored in the memory 92, for example, the ultrasound image generation method provided in any of the above embodiments.

The electronic device 90 may also communicate with one or more external devices 94 (e.g., a keyboard, a pointing device, etc.). Such communication may be performed through an input/output (I/O) interface 95. Furthermore, the electronic device 90 may also communicate with one or more networks (e.g., a local area network (LAN), a wide area network (WAN), and/or a public network, such as the Internet) through a network adapter 96. As shown in the figure, the network adapter 96 communicates with other modules of the electronic device 90 via the bus 93. It should be understood that although not shown in the figure, other hardware and/or software modules may be used in conjunction with the electronic device 90, including but not limited to: microcode, device drivers, redundant processors, external disk drive arrays, RAID (Redundant Array of Independent Disks) systems, tape drives, and data backup storage systems, etc.

It should be noted that although several units/modules or subunits/modules of the electronic device are mentioned in the detailed description above, such division is merely exemplary and not mandatory. Actually, according to the embodiments of the present disclosure, the features and functions of two or more units/modules described above may be embodied in one unit/module. Conversely, the features and functions of one unit/module described above may be further divided into multiple units/modules for embodiment.

Some embodiments of the present disclosure further provide a computer-readable storage medium on which a computer program is stored. When the program is executed by a processor, the ultrasound image generation method provided in any of the above embodiments is implemented.

The readable storage medium may more specifically include, but is not limited to: a portable disk, a hard disk, a random access memory, a read-only memory, an erasable programmable read-only memory, an optical storage device, a magnetic storage device, or any suitable combination thereof.

Some embodiments of the present disclosure further provide a computer program product, comprising a computer program. When the computer program is executed by a processor, the ultrasound image generation method described in any of the above embodiments is implemented. The program code for executing the computer program product of the present disclosure may be written in any combination of one or more programming languages. The program code may be executed entirely on a user device, partially on a user device, as a standalone software package, partially on a user device and partially on a remote device, or entirely on a remote device.

The basic concepts have been described above, apparently, in detail, as will be described above, and does not constitute limitations of the disclosure. Although there is no clear explanation here, those skilled in the art may make various modifications, improvements, and modifications of present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. As in “an embodiment”, “an embodiment”, and/or “some embodiments” means a feature, structure, or characteristic associated with at least one embodiment of the present application. Therefore, it is emphasized and should be appreciated that two or more references to “an embodiment” or “one embodiment” or “an alternative embodiment” in various parts of present disclosure are not necessarily all referring to the same embodiment. In addition, some features, structures, or features in the present disclosure of one or more embodiments may be appropriately combined.

Moreover, unless the claims are clearly stated, the sequence of the present disclosure, the use of the digital letters, or the use of other names is not configured to define the order of the present disclosure processes and methods. Although some examples of the disclosure currently considered useful in the present disclosure are discussed in the above disclosure, it should be understood that the details will only be described, and the appended claims are not limited to the disclosure embodiments. The requirements are designed to cover all modifications and equivalents combined with the substance and range of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

Some embodiments use numbers to describe the number of components, attributes, and it should be understood that such numbers used in the description of the embodiments are modified in some examples by the modifiers “about”, “approximately”, or “substantially”. Unless otherwise noted, the terms “about,” “approximately,” or “approximately” indicates that a ±20% variation in the stated number is allowed. Correspondingly, in some embodiments, the numerical parameters used in the specification and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present application are approximations, in specific embodiments such values are set to be as precise as possible within a feasible range.

For each patent, patent application, patent application disclosure, and other material cited in this application, such as articles, books, specifications, publications, documents, etc., the entire contents of which are hereby incorporated herein by reference. Except for application history documents that are inconsistent with or conflict with the contents of this application, and except for documents (currently or hereafter appended to this application) that limit the broadest scope of the claims of this application. It should be noted that in the event of any inconsistency or conflict between the descriptions, definitions, and/or use of terms in the materials appurtenant to this application and those set forth herein, the descriptions, definitions, and/or use of terms in this application shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. An ultrasound image generation method, comprising:
obtaining a plurality of ultrasonic echo signals of a subject, wherein the plurality of ultrasonic echo signals are acquired based on one or more receiving planes corresponding to one or more transmitting planes;
obtaining a plurality of receiving plane estimations corresponding to the one or more receiving planes;
generating a plurality of ultrasonic echo signal estimations each of which corresponds to one of the plurality of receiving plane estimations based on the plurality of ultrasonic echo signals;
generating a two-dimensional ultrasound image of each of the plurality of receiving plane estimations based on the plurality of ultrasonic echo signal estimations corresponding to the plurality of receiving plane estimations; and
obtaining a three-dimensional ultrasound image of the subject based on two-dimensional ultrasound images of the plurality of receiving plane estimations.

2. The method according to claim 1, wherein the obtaining a plurality of receiving plane estimations corresponding to the one or more receiving planes includes:
determining the plurality of receiving plane estimations corresponding to each of the one or more receiving planes based on a configuration parameter of an ultrasound device.

3. The method according to claim 2, wherein the determining the plurality of receiving plane estimations corresponding to each of the one or more receiving planes based on a configuration parameter of an ultrasound device includes:
determining, based on the configuration parameter, a second count of the plurality of receiving plane estimations corresponding to each of the one or more receiving planes, wherein the plurality of receiving plane estimations include a plurality of different virtual receiving planes sequentially arranged along two sides of a receiving plane corresponding to the each of the transmitting planes; and
determining the plurality of receiving plane estimations corresponding to each of the one or more receiving planes based on the second count of the plurality of receiving plane estimations.

4. The method according to claim 3, wherein the determining a second count of receiving plane estimations corresponding to each of the one or more receiving planes based on the configuration parameter includes:
determining an included angle between each of the plurality of different virtual receiving planes and the receiving plane based on an elevation angle spread of the receiving plane and the second count of the receiving plane estimations; wherein the elevation angle spread represents an included angle between adjacent receiving planes; and
determining the plurality of the virtual receiving planes arranged along the two sides of the receiving plane based on the included angle.

5. The method according to claim 3, wherein the determining a second count of the plurality of receiving plane estimations corresponding to each of the one or more receiving planes based on the configuration parameter includes:
in response to a volume of the subject being greater than a preset volume, increasing an initial second count of the plurality of receiving plane estimations by adjusting the configuration parameter to obtain the second count.

6. The method according to claim 3, wherein the determining a second count of the plurality of receiving plane estimations corresponding to each of the one or more receiving planes based on the configuration parameter includes:
in response to a heartbeat frequency of the subject being greater than a preset frequency, reducing an initial second count of the plurality of receiving plane estimations by adjusting the configuration parameter to obtain the second count.

7. The method according to claim 1, wherein the obtaining a plurality of receiving plane estimations corresponding to the one or more receiving planes includes:
determining a count of the plurality of receiving plane estimations corresponding to the each of the one or more receiving planes based on a user input parameter; and
determining the plurality of receiving plane estimations corresponding to the each of the plurality of receiving planes based on the count of the plurality of receiving plane estimations.

8. The method according to claim 1, wherein the generating a plurality of ultrasonic echo signal estimations each of which corresponds to one of the plurality of receiving plane estimations includes:
processing one or more ultrasonic echo signals corresponding to one of the receiving planes using a beamforming algorithm to obtain the plurality of ultrasonic echo signal estimations of the ultrasonic echo signal corresponding to the plurality of receiving plane estimations.

9. The method according to claim 1, wherein the generating a two-dimensional ultrasound image of each of the receiving plane estimations based on the plurality of ultrasonic echo signal estimations includes:
in response to an overlap of receiving plane estimations corresponding to different receiving planes among the one or more receiving planes, obtaining ultrasonic echo signal estimations corresponding to the receiving plane estimations at an overlapping position of the receiving plane estimations;
performing superposition processing on the ultrasonic echo signal estimations at the overlapping position using a superposition algorithm to obtain a superposed ultrasonic echo signal estimation;
generating a two-dimensional ultrasound image corresponding to the overlapping position based on the superposed ultrasonic echo signal estimation.

10. The method according to claim 9, wherein the performing superposition processing on the ultrasonic echo signal estimations at the overlapping position using a superposition algorithm to obtain a superposed ultrasonic echo signal estimation includes:
performing a coherent superposition processing or an incoherent superposition processing on the ultrasonic echo signal estimations at the overlap position based on weight values corresponding to different ultrasonic echo signal estimations at the overlapping position to obtain the superposed ultrasonic echo signal estimation;
wherein the weight values corresponding to the ultrasonic echo signal estimations are obtained based on an included angle between the receiving plane estimations at the overlapping position and the corresponding receiving planes corresponding to the receiving plane estimations at the overlapping position.

11. The method according to claim 1, wherein the generating a two-dimensional ultrasound image of each of the plurality of receiving plane estimations based on the plurality of ultrasonic echo signal estimations includes:
in response to determining that a target receiving plane estimation does not overlap with other receiving plane estimations among the plurality of receiving plane estimations,
obtaining each of target ultrasonic echo signal estimations corresponding to the target receiving plane estimation, and
generating the two-dimensional ultrasound image of each of the target receiving plane estimations based on the target ultrasonic echo signal estimations.

12. The method according to claim 1, wherein before obtaining a three-dimensional ultrasound image of the subject based on two-dimensional ultrasound images corresponding to the one or more receiving planes, the method further includes:
in response to determining that receiving plane estimations corresponding to different receiving planes among the one or more receiving planes overlaps, obtaining two-dimensional ultrasound images corresponding to the receiving plane estimations at an overlapping position of the receiving plane estimations; and
performing a superposition processing on the two-dimensional ultrasound images at the overlapping position using an incoherent superposition algorithm to obtain an updated two-dimensional ultrasound image corresponding to the overlapping position.

13. The method according to claim 1, wherein the obtaining a three-dimensional ultrasound image of the subject based on the two-dimensional ultrasound images includes:
processing the two-dimensional ultrasound images using a filtering algorithm and a three-dimensional interpolation algorithm to generate the three-dimensional ultrasound image of the subject.

14. The method according to claim 13, wherein the three-dimensional interpolation algorithm includes two or more interpolation processes.

15. The method according to claim 1, wherein the method further comprises:
obtaining the three-dimensional ultrasound image of the subject at different acquisition times to generate a four-dimensional ultrasound image of the subject.

16. An ultrasound image generation system, comprising:
at least one storage device, comprising an instruction set; and
at least one processor in communication with the at least one storage device, wherein when the instruction set is executed, the at least one processor is configured to perform steps comprising:
obtaining a plurality of ultrasonic echo signals of a subject, wherein the plurality of ultrasonic echo signals are acquired based on one or more receiving planes corresponding to one or more transmitting planes;
obtaining a plurality of receiving plane estimations corresponding to the one or more receiving planes;
generating a plurality of ultrasonic echo signal estimations each of which corresponds to one of the plurality of receiving plane estimations based on the plurality of ultrasonic echo signals;
generating a two-dimensional ultrasound image of each of the plurality of receiving plane estimations based on the plurality of ultrasonic echo signal estimations corresponding to the plurality of receiving plane estimations; and
obtaining a three-dimensional ultrasound image of the subject based on two-dimensional ultrasound images of the plurality of receiving plane estimations.

17. The system according to claim 16, wherein the obtaining a plurality of receiving plane estimations corresponding to the receiving planes includes:
determining the plurality of receiving plane estimations corresponding to each of the receiving planes based on a configuration parameter of an ultrasound device.

18. A computer-readable storage medium, storing computer instructions, wherein when a computer reads the computer instructions from the computer-readable storage medium, the computer executes the ultrasound image generation method according to claim 1.

19. An ultrasound image generation method, comprising:
obtaining a plurality of sets of ultrasonic echo signals of a subject, each set of the ultrasonic echo signals being acquired via one transmission pulse;
for each of at least one set of the plurality of sets of ultrasonic echo signals, generating a plurality of sets of ultrasonic echo signal estimations based on the each of at least one set of the plurality of sets of ultrasonic echo signals;
determining a plurality of 2D images based on the plurality of sets of ultrasonic echo signal estimations for the each of at least one set of the plurality of sets of ultrasonic echo signals;
determining a 3D image based on the plurality of 2D images.

20. The ultrasound image generation method of the claim 19, wherein the generating a plurality of sets of ultrasonic echo signal estimations based on the each of at least one set of the plurality of sets of ultrasonic echo signals includes:
processing each of the plurality of sets of ultrasonic echo signals using a beamforming algorithm to obtain the plurality of sets of ultrasonic echo signal estimations corresponding to each of the plurality of sets of ultrasonic echo signals.
